SEQUENCE LISTING (7 sequences)

SEQ ID NO: 1 - DNA, 27 bp, Homo sapiens
  modified_base 1: mod_base = OTHER; note = n is Hexachlorofluorescein-deoxyadenosine monophosphate
  modified_base 27: mod_base = OTHER; note = n is Carboxytetramethylrhodamine-dideoxycytosine monophosphate
  source 1..27: mol_type = other DNA
nctctgagccctctttccaaactactn

SEQ ID NO: 2 - DNA, 27 bp, Homo sapiens
  modified_base 27: mod_base = OTHER; note = n is Dideoxycytosine monophosphate
  source 1..27: mol_type = other DNA
actctgagccctctttccaaactactn

SEQ ID NO: 3 - DNA, 28 bp, Aids-associated retrovirus
  modified_base 11: mod_base = OTHER; note = n is Carboxyfluorescein-deoxythymidine monophosphate
  modified_base 28: mod_base = OTHER; note = n is Carboxytetramethylrhodamine-dideoxyuridine monophosphate
  source 1..28: mol_type = other DNA
ttggaggacancaagcagccatgcaaan

SEQ ID NO: 4 - DNA, 28 bp, Aids-associated retrovirus
  modified_base 1: mod_base = OTHER; note = n is Carboxyfluorescein-deoxythymidine monophosphate
  modified_base 28: mod_base = OTHER; note = n is Carboxytetramethylrhodamine-dideoxyuridine monophosphate
  source 1..28: mol_type = other DNA
ntggaggacatcaagcagccatgcaaan

SEQ ID NO: 5 - DNA, 27 bp, Aids-associated retrovirus
  modified_base 27: mod_base = OTHER; note = n is Dideoxythymidine monophosphate
  source 1..27: mol_type = other DNA
tgctatgtcagttccccttggttctcn

SEQ ID NO: 6 - DNA, 28 bp, Homo sapiens
  modified_base 28: mod_base = OTHER; note = n is Dideoxycytosine monophosphate
  source 1..28: mol_type = other DNA
gtctcaaagattccagaagtcagtacan

SEQ ID NO: 7 - DNA, 28 bp, Homo sapiens
  modified_base 28: mod_base = OTHER; note = n is Dideoxycytosine monophosphate
  source 1..28: mol_type = other DNA
gtctcaaagattccagaagtcaggaaan

(19) United States
(12) Patent Application Publication
(10) Pub. No.: US 2024/0209428 A1
(43) Pub. Date: Jun. 27, 2024

(54) Pyrophosphorolysis activated fluorescence to measure PAP amplification of nucleic acid

(71) Applicants: Shaofeng Ding, Santa Fe Springs, CA (US); Qiang Liu, Rancho Cucamonga, CA (US)

(72) Inventors: Shaofeng Ding, Santa Fe Springs, CA (US); Qiang Liu, Rancho Cucamonga, CA (US)

(21) Appl. No.: 18/426,132

(22) Filed: Jan. 29, 2024

Related U.S. Application Data
(63) Continuation-in-part of application No. 16/658,029, filed on Oct. 19, 2019, now Pat. No. 11,921,049.

(51) Int. Cl. C12Q 1/6853

(52) U.S. Cl. CPC C12Q 1/6853; C12Q 2600/16

(57) ABSTRACT

A new fluorescence detection method called pyrophosphorolysis activated fluorescence was developed to measure PAP amplification of nucleic acid. A fluorophore-quencher-dual-labeled blocked primer was used for PAP: I) which has a fluorophore attached to a nucleotide in the internal region or at the 5′ end and a quencher attached to a blocked nucleotide at the 3′ end, or II) which has a quencher attached to a nucleotide in the internal region or at the 5′ end and a fluorophore attached to a blocked nucleotide at the 3′ end. Multiple fluorophore-quencher-dual-labeled blocked primers were also used for multiplex PAP, which are attached with different fluorophores to distinguish multiple templates in a reaction.

### CROSS-REFERENCE TO RELATED APPLICATIONS

This US non-provisional patent application claims priority from U.S. non-provisional patent application Ser. No. 16/658,029, filed on Oct. 19, 2019, and further claims priority from U.S. provisional patent application No. 62/757,767, filed on Nov. 9, 2018, each of which is incorporated by reference in its entirety.

### SEQUENCE LISTING

This application is being filed along with a Sequence Listing and its electronic format entitled SequenceListing.txt.

### BACKGROUND OF THE INVENTION

### Field of the Invention

The present invention relates to the field of molecular biology and particularly pyrophosphorolysis activated polymerization (PAP) for nucleic acid amplification.

### Description of the Prior Art

### Pyrophosphorolysis Activated Polymerization (PAP)

Pyrophosphorolysis activated polymerization (PAP) is a method for nucleic acid amplification where pyrophosphorolysis and polymerization are serially coupled by DNA polymerase using 3′ blocked primers (Liu and Sommer, 2000; Liu and Sommer, 2004b). A primer is blocked at the 3′ end with a non-extendable nucleotide (3′ blocker), such as a dideoxynucleotide, and cannot be directly extended by DNA polymerase. When the 3′ blocked primer anneals to its complementary DNA template, DNA polymerase can remove the 3′ blocker from the 3′ blocked primer in the presence of pyrophosphate or its analog, which reaction is called pyrophosphorolysis. The DNA polymerase can then extend the 3′ unblocked primer on the DNA template. In addition to references cited herein, PAP has been described in U.S. Pat. Nos. 6,534,269, 7,033,763, 7,105,298, 7,238,480, 7,504,221, 7,914,995, and 7,919,253.

The serial coupling of pyrophosphorolysis and extension using the 3′ blocked primer in PAP results in an extremely high selectivity (Liu and Sommer, 2004a; Liu and Sommer, 2004b) because a significant nonspecific amplification (Type II error) requires mismatch pyrophosphorolysis followed by mis-incorporation by the DNA polymerase, an event with a frequency estimated to be 3.3×10⁻¹¹.

The bi-directional form of PAP (Bi-PAP) is especially suitable for allele-specific amplification that uses two opposing 3′ blocked primers with a single nucleotide overlap at their 3′ ends (Liu and Sommer, 2004a; Liu and Sommer, 2004b). Bi-PAP can detect one copy of a mutant allele in the presence of 10⁹ copies of the wild type DNA without false positive amplifications.

### DNA-PAP

PAP was initially tested with Tfl and Taq polymerases using DNA template of the human dopamine D1 gene, proving the principle that DNA-dependent DNA pyrophosphorolysis and DNA-dependent DNA polymerization can be serially coupled (Liu and Sommer, 2000). The efficiency of PAP was greatly improved using TaqFS, a genetically engineered polymerase comprising a F667Y mutation, which were demonstrated using other DNA templates (Liu and Sommer, 2002).

### RNA-PAP (RT-PAP)

RT-PAP was developed that can directly amplify RNA template without additional treatment. RT-PAP brings in a new mechanism for amplification of RNA template in which RNA-dependent DNA pyrophosphorolysis removes 3′ blocker such as 3′ dideoxynucleotide from a blocked primer when hybridized to RNA template, and then the RNA-dependent DNA polymerization extends the activated primer. Due to this serial coupling, RT-PAP has high selectivity against mismatches on the RNA template, providing highly specific amplification of RNA template (U.S. Pat. No. 9,133,491).

PAP with Acycolonucleotide Blocker and Type II Polymerase

We showed that Type II DNA polymerase efficiently catalyzes template-dependent pyrophosphorolysis to activate primers blocked at their 3′ termini with acyclonucleotides in which a 2-hydroxyethoxymethyl group substitutes for the 2′-deoxyribofuranosyl sugar. Type II DNA polymerases Vent (exo-) and Pfu (exo-) were used for PAP with acyclonucleotide-blocked primers, besides Type I DNA polymerase (Liu and Sommer, 2004c).

### Multiplex-PAP

Advantageous to produce little or no primer-dimer or false priming (Liu and Sommer, 2002), multiple pairs of primers (≥2) were used to amplify multiple potential templates (≥2) located at multiple loci (≥2) in one reaction (Liu, et al., 2006). In an example, multiplex PAP used eight pairs of primers that targeted eight loci in human genome including seven different exons scattered along a 30 Kb sequence of the human factor IX gene and one exon in the human ATM gene.

Multiplex PAP can also uses multiple pairs of blocked primers to amplify multiple almost-sequence-identical templates located in one locus in a single reaction, among which the sequence differences may be as little as one base substitution, a few base deletion or insertion, such as in the KRAS gene (US patent application publication 20180265919).

### Current Fluorescence Methods to Measure Amplification of Nucleic Acid

Currently, fluorescent chemistries available can be categorized into two major types: 1) DNA-binding dyes (such as SYBR Green I), and 2) dye-labeled, sequence-specific oligonucleotide primers or probes (such as TaqMan probe).

SYBR Green I exhibits little fluorescence when free in solution, but its emitted fluorescence increases up to 1,000-fold intensity when binds nonspecifically to double-stranded DNA.

The advantages of SYBR Green I include simple assay design (probe is not necessary), lower cost, and melt-curve analysis to check specific amplified product. However, a major drawback is non-specific binding to double-stranded DNA, such as dimmers, and non-multiplexing.

TaqMan probe is typically an oligonucleotide with a fluorophore covalently attached to a nucleotide at the 5′ end, and a quencher to a nucleotide at the 3′ end. The quencher molecule quenches the fluorescence emitted by the fluorophore through a mechanism of Förster resonance energy transfer (FRET) (Holland et al., 1991).

In PCR amplification, when Taq polymerase extends a primer, its 5′-3′ exonuclease activity hydrolyzes the probe once annealed to its template. Thus, the hydrolysis releases the fluorophore from the quencher to emit fluorescence.

The main advantages of TaqMan probe include high specificity, high signal-to-noise ratio, and multiplexing because multiple probes can have different fluorophores to differentiate multiple templates in a single reaction.

### A New Fluorescence Detection Method and its Advantages

A new method of pyrophosphorolysis activated fluorescence was developed to measure PAP amplification of nucleic acid. A single fluorophore-quencher-dual-labeled blocked primer was used for singleplex PAP in two forms: I) which has a fluorophore attached to a nucleotide in the internal region or at the 5′ end and a quencher attached to a blocked nucleotide at the 3′ end, or II) which has a quencher attached to a nucleotide in the internal region or at the 5′ end and a fluorophore attached to a blocked nucleotide at the 3′ end.

Multiple fluorophore-quencher-dual-labeled blocked primers were used for multiplex PAP, which are attached with different fluorophores to distinguish multiple templates in a reaction. In Multiplex-PAP, the use of one universal-quencher but of multiple fluorophores simplifies the primer design and improves the amplification uniformity.

### SUMMARY OF THE INVENTION

### Form I of Fluorophore-Quencher-Dual-Labeled Blocked Primer:

For a pair of forward and reverse blocked primers (Form I) for pyrophosphorolysis activated polymerization comprises a 3′ blocked primer to be complementary to a template, in which a fluorophore is covalently attached to a nucleotide in the internal region or at the 5′ end and a quencher is covalently attached to the base of a non-extendable, blocked nucleotide that lacks the 3′ hydroxyl group and is located at the 3′ end of the primer (FIG. 1A).

Once the primer anneals to its template, the 3′ non-extendable, blocked nucleotide attached with the quencher is removed from the 3′ end of the primer by pyrophosphorolysis catalyzed by a polymerase to generate two products of 1) a 3′ unblocked primer which is covalently labeled with the fluorophore in the internal region or at the 5′ end, and 2) a corresponding blocked nucleoside triphosphate which base is covalently labeled with the quencher, thus the fluorophore emits a fluorescence signal in PAP amplification (FIG. 1A).

The forward or reverse blocked primer has a FAM (carboxyfluorescein) or HEX (hexachlorofluorescein) fluorophore attached to a nucleotide in the internal region or at the 5′ end and a TAMRA (carboxytetramethylrhodamine) quencher attached to a non-extendable, blocked nucleotide at the 3′ end.

Once the non-extendable, blocked nucleotide at the 3′ end attached with the TAMRA (carboxytetramethylrhodamine) quencher is removed by pyrophosphorolysis, the FAM (carboxyfluorescein) or HEX (hexachlorofluorescein) fluorophore generates a detectable fluorescence signal in PAP amplification.

The non-extendable, blocked nucleotide at the 3′ end is a dideoxynucleotide or an acyclonucleotide.

Of the forward or reverse blocked primer, emission spectrum of the fluorophore overlaps absorbance spectrum of the quencher.

Of the forward or reverse blocked primer, the fluorophore is 30 bases or less from the 3′ end.

The forward or reverse blocked primer has an artificial mutation introduced into the 3′ region, which can delay the product accumulation to a later time or cycle in a method called delayed-PAP.

The artificial mutation is a single base substitution.

The artificial mutation ranges from the 3^{rd} base to the 5th base from the 3′ end.

A plurality of 3′ blocked primers (Form I) for multiplex pyrophosphorolysis activated polymerization comprise:
a) a first 3′ blocked primer to be complementary to a first template, in which a first fluorophore is covalently attached to a nucleotide in the internal region or at the 5′ end and a universal quencher is covalently attached to the base of a first non-extendable, blocked nucleotide that lacks the 3′ hydroxyl group and is located at the 3′ end of the first primer, and in which once the first primer anneals to the first template, the first 3′ non-extendable, blocked nucleotide attached with the universal quencher is removed from the 3′ end of the first primer by pyrophosphorolysis catalyzed by a polymerase to generate two products of 1) a first 3′ unblocked primer which is covalently labeled with the first fluorophore in the internal region or at the 5′ end, and 2) a first corresponding blocked nucleoside triphosphate which base is covalently labeled with the universal quencher, thus the first fluorophore emits a fluorescence signal in PAP amplification, and
b) a second 3′ blocked primer to be complementary to a second template, in which a second fluorophore is covalently attached to a nucleotide in the internal region or at the 5′ end and the same universal quencher is attached to the base of a second non-extendable, blocked nucleotide which lacks the 3′ hydroxyl group and is located at the 3′ end of the second primer, and in which once the second primer anneals to the second template, the second 3′ non-extendable, blocked nucleotide covalently attached with the same universal quencher is removed from the 3′ end of the second primer by pyrophosphorolysis catalyzed by the polymerase to generate two products of 1) a second 3′ unblocked primer which is covalently labeled with the second fluorophore in the internal region or at the 5′ end, and 2) a second corresponding blocked nucleoside triphosphate which base is covalently labeled with the universal quencher, thus the second fluorophore emits another fluorescence signal in PAP amplification.

The first blocked primer has a FAM (carboxyfluorescein) fluorophore attached to a nucleotide in the internal region or at the 5′ end and a universal TAMRA (carboxytetramethylrhodamine) quencher attached to a first non-extendable, blocked nucleotide at the 3′ end, and the second blocked primer has a HEX (hexachlorofluorescein) fluorophore attached to a nucleotide in the internal region or at the 5′ end and the same universal TAMRA quencher attached to the second non-extendable, blocked nucleotide at the 3′ end.

The non-extendable, blocked nucleotides at the 3′ ends are dideoxynucleotides or acyclonucleotides.

Of the plurality of 3′ blocked primers, the first primer has the same universal quencher as the second primer, but the first primer has a different fluorophore.

Of the plurality of 3′ blocked primers, the first template is an internal control and the second template is a target.

Of the plurality of 3′ blocked primers, emission spectrums of the fluorophores overlap an absorbance spectrum of the universal quencher.

Of the plurality of 3′ blocked primers, the fluorophores are located 30 bases or less from the 3′ ends.

A method for multiplex pyrophosphorolysis activated polymerization (Form I) with a plurality of blocked primers comprises:
a) first 3′ blocked primer to be complementary to a first template, in which a first fluorophore is covalently attached to a nucleotide in the internal region or at the 5′ end and a universal quencher is covalently attached to the base of a first non-extendable, blocked nucleotide that lacks the 3′ hydroxyl group and is located at the 3′ end of the primer,
b) once the first primer anneals to the first template, the first 3′ non-extendable, blocked nucleotide attached with the universal quencher is removed from the 3′ end of the first primer by pyrophosphorolysis catalyzed by a polymerase to generate two products of 1) a first 3′ unblocked primer which is covalently labeled with the first fluorophore in the internal region or at the 5′ end, and 2) a first corresponding blocked nucleoside triphosphate which base is covalently labeled with the universal quencher, thus the first fluorophore emits a fluorescence signal in PAP amplification,
c) a second 3′ blocked primer to be complementary to a second template, in which a second fluorophore is covalently attached to a nucleotide in the internal region or at the 5′ end and the same universal quencher is attached to the base of a second non-extendable, blocked nucleotide which lacks the 3′ hydroxyl group and is located at the 3′ end of the primer, and
d) once the second primer anneals to the second template, the second 3′ non-extendable, blocked nucleotide covalently attached with the same universal quencher is removed from the 3′ end of the second primer by pyrophosphorolysis catalyzed by the polymerase to generate two products of 1) a second 3′ unblocked primer which is covalently labeled with the second fluorophore in the internal region or at the 5′ end, and 2) a second corresponding blocked nucleoside triphosphate which base is covalently labeled with the universal quencher, thus the second fluorophore emits another fluorescence signal in PAP amplification.

### Form II of Fluorophore-Quencher-Dual-Labeled Blocked Primer:

A pair of forward and reverse blocked primers (Form II) for pyrophosphorolysis activated polymerization comprise a 3′ blocked primer to be complementary to the template, in which a quencher is covalently attached to a nucleotide in the internal region or at the 5′ end and a fluorophore is covalently attached to the base of a non-extendable, blocked nucleotide that lacks the 3′ hydroxyl group and is located at the 3′ end of the primer (FIG. 1B).

Once the primer anneals to its template, the 3′ non-extendable, blocked nucleotide attached with the fluorophore is removed from the 3′ end of the primer by pyrophosphorolysis catalyzed by a polymerase to generate two products of 1) a 3′ unblocked primer which is covalently labeled with the quencher in the internal region or at the 5′ end, and 2) a corresponding blocked nucleoside triphosphate which base is covalently labeled with the fluorophore, thus the fluorophore emits a fluorescence signal in PAP amplification (FIG. 1B).

The 3′ blocked primer has a FAM (carboxyfluorescein) or HEX (hexachlorofluorescein) fluorophore attached to blocked nucleotide at the 3′ end and a TAMRA (carboxytetramethylrhodamine) quencher to a nucleotide in the internal region or at the 5′ end.

Of the 3′ blocked primers, the emission spectrums of the fluorophores overlap an absorbance spectrum of the quencher.

Of the 3′ blocked primers, the blocked nucleotide at the 3′ end is a dideoxynucleotide or an acyclonucleotide.

A plurality of 3′ blocked primers (Form II) for multiplex pyrophosphorolysis activated polymerization comprise:
a) a first 3′ blocked primer to be complementary to a first template, in which a universal quencher is covalently attached to a nucleotide in the internal region or at the 5′ end, and a first fluorophore is covalently attached to the base of a first non-extendable, blocked nucleotide that lacks the 3′ hydroxyl group and is located at the 3′ end of the primer, and in which once the first primer anneals to the first template, the first 3′ non-extendable, blocked nucleotide attached with the first fluorophore is removed from the 3′ end of the first primer by pyrophosphorolysis catalyzed by a polymerase to generate two products of 1) a first 3′ unblocked primer which is covalently labeled with the universal quencher in the internal region or at the 5′ end, and 2) a first corresponding blocked nucleoside triphosphate which base is covalently labeled with the first fluorophore, thus the first fluorophore emits a fluorescence signal in PAP amplification, and
b) a second 3′ blocked primer to be complementary to a second template, in which the universal quencher is covalently attached to a nucleotide in the internal region or at the 5′ end, a second fluorophore is covalently attached to the base of a second non-extendable, blocked nucleotide that lacks the 3′ hydroxyl group and is located at the 3′ end of the primer, and in which once the second primer anneals to the second template, the second 3′ non-extendable, blocked nucleotide covalently attached with the second fluorophore is removed from the 3′ end of the second primer by pyrophosphorolysis catalyzed by the polymerase to generate two products of 1) a second 3′ unblocked primer which is covalently labeled with the universal quencher in the internal region or at the 5′ end, and 2) a second corresponding blocked nucleoside triphosphate which base is covalently labeled with the second fluorophore, thus the second fluorophore emits another fluorescence signal in PAP amplification.

Of the plurality of 3′ blocked primers, the first blocked primer has a FAM (carboxyfluorescein) fluorophore attached to the first blocked nucleotide at the 3′ end, and a universal TAMRA (carboxytetramethylrhodamine) quencher attached to the nucleotide in the internal region or at the 5′ end and, and the second blocked primer has the same universal TAMRA quencher attached to a nucleotide in the internal region or at the 5′ end, and a HEX (hexachlorofluorescein) fluorophore attached to the second blocked nucleotide at the 3′ end.

Of the plurality of 3′ blocked primers, the first primer has the same universal quencher as the second primer, but the first primer has a different fluorophore with the second primer.

Of the plurality of 3′ blocked primers, the first template is an internal control and the second template is a target.

Of the plurality of 3′ blocked primers, the emission spectrums of the fluorophores overlap an absorbance spectrum of the universal quencher.

Of the plurality of 3′ blocked primers, the quencher is located 30 bases or less from the 3′ ends.

Of the plurality of 3′ blocked primers, the first blocked nucleotide is an acyclonucleotide, and the second blocked nucleotide is an acyclonucleotide.

A method for multiplex pyrophosphorolysis activated polymerization with a plurality blocked primers (Form II) comprises:
a) a first 3′ blocked primer to be complementary to a first template, in which a universal quencher is covalently attached to a nucleotide in the internal region or at the 5′ end, and a first fluorophore is covalently attached to the base of a first non-extendable, blocked nucleotide that lacks the 3′ hydroxyl group and is located at the 3′ end of the primer,
b) once the first primer anneals to the first template, the first 3′ non-extendable, blocked nucleotide attached with the first fluorophore is removed from the 3′ end of the first primer by pyrophosphorolysis catalyzed by a polymerase to generate two products of 1) a 3′ first unblocked primer which is covalently labeled with the universal quencher in the internal region or at the 5′ end, and 2) a first corresponding blocked nucleoside triphosphate which base is covalently labeled with the first fluorophore, thus the first fluorophore emits a fluorescence signal in PAP amplification,
c) a second 3′ blocked primer to be complementary to a second template, in which the universal quencher is covalently attached to a nucleotide in the internal region or at the 5′ end, a second fluorophore is covalently attached to the base of a second non-extendable, blocked nucleotide that lacks the 3′ hydroxyl group and is located at the 3′ end of the primer, and
d) once the second primer anneals to the second template, the second 3′ non-extendable, blocked nucleotide covalently attached with the second fluorophore is removed from the 3′ end of the second primer by pyrophosphorolysis catalyzed by the polymerase to generate two products of 1) a second 3′ unblocked primer which is covalently labeled with the universal quencher in the internal region or at the 5′ end, and 2) a second corresponding blocked nucleoside triphosphate which base is covalently labeled with the second fluorophore, thus the second fluorophore emits another fluorescence signal in PAP amplification.

### BRIEF DESCRIPTION OF THE DRAWINGS

FIG. 1. Principle of pyrophosphorolysis activated fluorescence

Panel A shows format I of fluorophore-quencher-dual-labeled blocked primer. A fluorophore (F), such as FAM, is covalently attached to a nucleotide in the internal region or at the 5′ end, and a quencher (Q), such as TAMRA, is covalently attached to a blocked nucleotide at the 3 end, such as dideoxynucleotide. In PAP amplification, once the primer anneals to its complementary template, pyrophosphorolysis removes the 3′ non-extendable, blocked nucleotide covalently attached with the quencher, releasing the fluorophore from quencher to emit fluorescence (indicated by a star symbol), and then polymerization extends the unblocked primer.

Panel B shows format II of fluorophore-quencher-dual-labeled blocked primer. A fluorophore (F) is covalently attached to a blocked nucleotide at the 3′ end and a quencher (Q) is covalently attached to a nucleotide in the internal region or at the 5′ end.

FIG. 2. Pyrophosphorolysis activated fluorescence in simpleplex and multiplex PAP assays

Panel A illustrates simpleplex PAP in the GNAS gene. The GNAS PAP assay includes a forward fluorophore-quencher-dual-labeled blocked primer (SEQ ID 1) and a reverse blocked primer (SEQ ID 2) (Table 2) in a reaction. In PAP amplification, a HEX fluorescence signal was generated and measured. The amplification plot is showed with the cycle number in X-axis and relative fluorescence unit in y-axis for the given cycle.

Panel B illustrates simpleplex PAP in the HIV gene. The HIV PAP assay includes a forward fluorophore-quencher-dual-labeled blocked primer (SEQ ID 3) and a reverse blocked primer (SEQ ID 5) (Table 2) in a reaction. In PAP amplification, a FAM fluorescence signal was generated and measured.

Panel C illustrates multiplex PAP in the GNAS and HIV genes. The multiplex PAP assay contains a first forward fluorophore-quencher-dual-labeled blocked primer and a first reverse blocked primer (SEQ ID 1 and 2) for the GNAS gene, and a second fluorophore-quencher-dual-labeled blocked primer and a second reverse blocked primer (SEQ ID 3 and 5) for the HIV gene in a reaction. In PAP amplification, HEX and FAM fluorescence signals were generated and measured.

### DETAILED DESCRIPTION OF THE INVENTION

### Terminology of PAP Technology

Unless defined otherwise, all technical and scientific terms used herein have the same meaning as commonly understood by one of ordinary skill in the art.

PCR refers to polymerase chain reaction.

Pyrophosphorolysis is the reverse reaction of deoxyribonucleic acid polymerization. In the presence of pyrophosphate, the 3′ nucleotide is removed by a polymerase from duplex DNA to generate a triphosphate nucleotide and a 3′ unblocked duplex DNA: [dNMP]ₙ+PPi→[dNMP]ₙ₋₁+dNTP (Deutscher and Kornberg, 1969).

Polymerase or nucleic acid polymerase refers to a polymerase characterized as polymerization or extension of deoxyribonucleic acids.

3′ blocked primer refers to an oligonucleotide with a 3′ non-extendable nucleotide (3′ blocker), such as a dideoxynucleotide or an acycolonucleotide. The 3′ nucleotide could not be directly extended, but it can be removed by pyrophosphorolysis and then the unblocked primer can be extended by polymerase.

PAP refers to pyrophosphorolysis activated polymerization.

Delayed pyrophosphorolysis activated polymerization (delayed-PAP) means that the product starts to accumulate at later time or cycle in the amplification process.

Bidirectional-PAP (Bi-PAP) is a form of PAP that uses a pair of opposing blocked primers that overlap by one nucleotide at their 30 termini.

Exponential-PAP is a form of PAP that uses a pair of two opposing forward and reverse primers for exponential product accumulation with cycles. At least one primer is blocked primer.

Sensitivity or detection limit is defined as the smallest copy number of a template that generates a detectable product when the blocked primers match the template at the targeted nucleotide, such as the 3′ end.

Specificity is defined as the largest copy number of a template that generates an undetectable product when the blocked primers mismatch the template at the targeted nucleotide, such as the 3′ end.

Selectivity, the ratio of sensitivity to specificity, is defined as the ability to detect a small number of copies of the matched template in the presence of a large number of copies of the mismatched template without causing false positives.

Thermostable enzyme refers to an enzyme that is heat stable or heat resistant.

TaqFS is a genetic engineered form of Taq polymerase containing G46E and F667Y amino acid changes compared with the wild type sequence. In PAP, it has 5′-3′ polymerase activity that can incorporate dNTP in extension, and pyrophosphorolysis activity that can remove ddNMP from the 3′ end of a primer, but no 5′-3′ exonuclease activity.

PAP polymerase is a genetic engineered form of Taq polymerase containing F667Y amino acid changes compared with the wild type sequence. It has 5′-3′ polymerase activity and pyrophosphorolysis activity.

A pair of primers means two opposing forward and reverse primers.

Singleplex PAP means that one pair of primers amplify one template in a reaction.

Multiplex PAP means that ≥2 pairs of primers amplify ≥2 potential templates in a reaction. The multiple templates may be located at multiple loci or at one locus. The sequence differences among the templates, may be as little as one base substitution, a few base deletion or insertion, and may be located as near as at the same nucleotide. In addition, the templates may be completely or partially overlapped within the region.

The 5′ region of a primer is the 5′ part of the primer sequence, such as the ten successive nucleotides from the 5′ end.

The 3′ region of a primer is the 3′ part of the primer sequence, such as the ten successive nucleotides from the 3′ end.

The internal region is the primer sequence between the 5′ end and the 3′ end.

The central region of a primer is the middle part of the primer sequence between the 5′ region and the 3′ region.

Starting template means the template which is present before amplification starts, such as those of plasmid and genomic DNA.

### Terminology of Fluorophore and Quencher

Fluorophore: a molecule, such as FAM, HEX, and TET, that displays fluorescence. The fluorescence is generated when the fluorophore absorbs light energy at a short wavelength and then emits light energy at a longer wavelength. Each fluorophore has a characteristic absorbance spectrum and a characteristic emission spectrum. The specific wavelength at which a fluorophore will most efficiently absorb energy is called the peak absorbance and the wavelength at which it will most efficiently emit energy is called the peak emission.

FAM is a fluorophore which has peak absorbance at 492 nm wavelength and peak emission at 520 nm wavelength.

HEX is a fluorophore which has peak absorbance at 535 nm wavelength and peak emission at 556 nm wavelength.

TET is a fluorophore which has peak absorbance at 521 nm wavelength and peak emission at 536 nm wavelength.

Quencher: a molecule, such as TAMRA, that decreases fluorescence intensity of a fluorophore report. It has a characteristic absorbance spectrum with a peak absorbance. For a quencher to function through a mechanism of a mechanism of fluorescence-resonance energy transfer (FRET), its absorbance spectrum overlaps the emission spectrum of the fluorophore, and its distance is proximate enough to the fluorophore, such as no more than 30 bases on a oligonucleotide.

TAMRA is a quencher which has peak absorbance 575 nm.

Universal quencher: a molecule, such as TAMRA, that decreases fluorescence intensities of two or more types of fluorophores.

Single fluorophore-labeled primer: a fluorophore, such as FAM, HEX, and TET, is covalently attached to a nucleotide at the 5′ end, or in the internal region, or at the 3′ end.

Single quencher-labeled primer: a quencher, such as TAMRA, is covalently attached to a nucleotide at the 5′ end, or in the internal region, or at the 3′ end.

Fluorophore-quencher-dual-labeled primer: there are two formats: 1) a fluorophore, such as FAM, HEX, and TET, is covalently attached to a nucleotide at the 5′ end or in the internal region, and a quencher, such as TAMRA, is covalently attached to a nucleotide at the 3′ end, and 2) a fluorophore, such as FAM, HEX, and TET, is covalently attached to a nucleotide at the 3′ end, and a quencher, such as TAMRA, is covalently attached to a nucleotide at the 5′ or in the internal region.

### Terminology of Real-Time Fluorescence Detection

Baseline is the level of fluorescence signal during initial cycles. The low level can be considered as background or “noise” of the reaction.

Threshold is defined as the level of fluorescence signal that is a significant higher than baseline signal and can distinguish amplification signal from the background.

Ct (threshold cycle) is the cycle number at which the fluorescence signal crosses the threshold.

### Terminology of Delayed-PAP

3′-perfect-match primer means the 3′ region has no artificial mutations and perfectly matches the starting template.

Artificial mutation means the mutation that is artificially introduced into primer sequences.

Artificial mismatch is formed between the artificial mutation in the 3′ region of a 3′-artificial-mutation primer and the complementary strand of a template.

3′-artificial-mutation primer means an artificial mutation is introduced into the 3′ region.

Regular-PAP means PAP with two 3′-perfect-match primers. PAP means regular-PAP unless stated otherwise.

Simplex regular-PAP means PAP with two 3′-perfect-match primers. Simplex PAP means simplex regular-PAP unless stated otherwise.

Multiplex regular-PAP means all PAP assays are regular-PAP assays in the multiplex format. Multiplex PAP means multiplex regular-PAP unless stated otherwise.

Delayed-PAP means PAP with one or two 3′-artificial-mutation primers to delay the product accumulation to a later time or cycle.

Multiplex delayed-PAP means at least one delayed-PAP assay in the multiplex format.

### Principle of Pyrophosphorolysis Activated Fluorescence to Measure PAP Amplification of Nucleic Acid

### 1) Fluorophore-Quencher-Dual-Labeled Blocked Primer

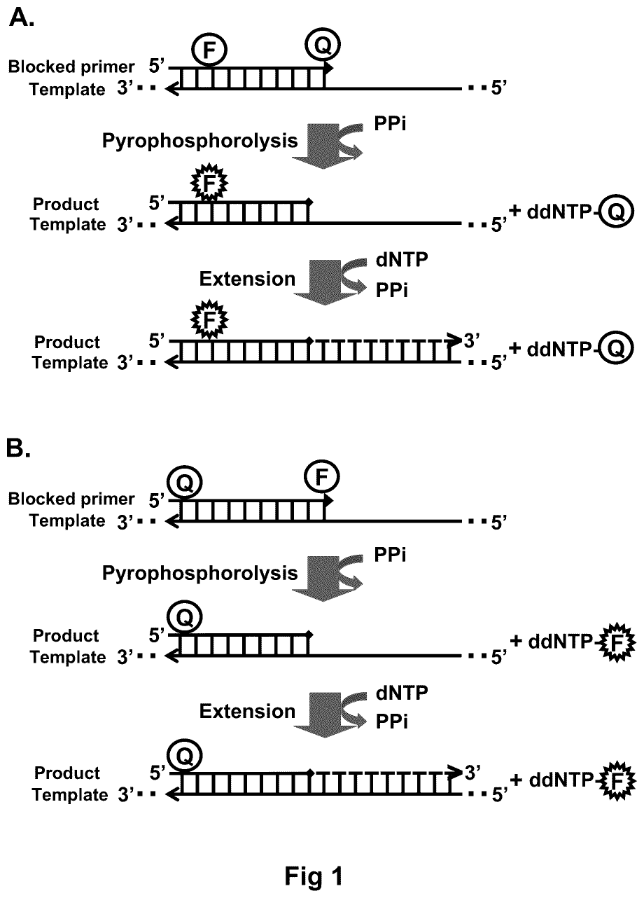

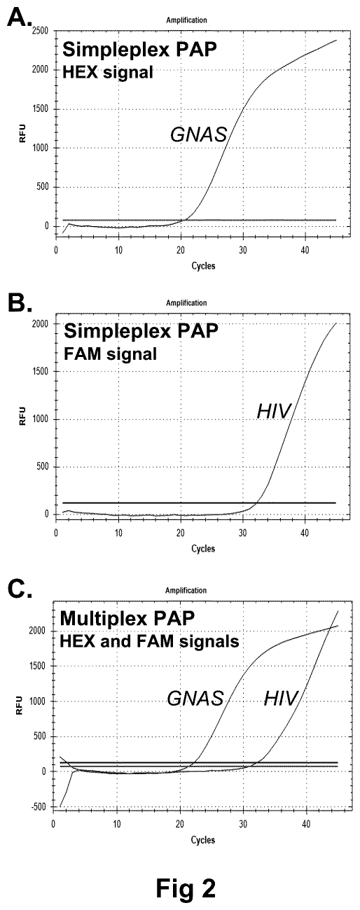

For format I of fluorophore-quencher-dual-labeled blocked primer, a fluorophore, such as FAM, HEX, or TET, is covalently attached to a nucleotide at the 5′ end or in the internal region, and a quencher, such as TAMRA, is covalently attached to a blocked nucleotide at the 3′ end, such as dideoxynucleotide (FIG. 1A).

The FAM, HEX, TET, and TAMRA have peak absorbances at 492 nm, 535 nm, 521 nm, 552 nm wavelengths, respectively; and peak emissions at 520 nm, 556 nm, 536 nm, 575 nm wavelengths, respectively. The absorbance spectrum of the TAMRA quencher overlaps the emission spectrums of the FAM, HEX, and TET fluorophores. Thus, multiple fluorophore-quencher-dual-labeled blocked primers may be labeled with different fluorophores but with the same universal quencher.

Other types of fluorophores, quenchers such as Dark Quenchers, and 3′ blocked nucleotides such as acyclonucleotide are also applicable, once they are satisfied by 1) the absorbance spectrum of the quencher overlaps the emission spectrum of the fluorophore, 2) the distance from the quencher to the fluorophore is ≤30 nucleotides so that the quencher can substantially quench the fluorophore, and 3) the 3′ blocked nucleotide covalently attached with the quencher can be removed by pyrophosphoresis in PAP.

For format II of fluorophore-quencher-dual-labeled blocked primer, a fluorophore, such as FAM, HEX, or TET, is covalently attached to a blocked nucleotide at the 3′ end, such as dideoxynucleotide, and a quencher, such as TAMRA, is covalently attached to a nucleotide at the 5′ or in the internal region (FIG. 1B).

In Multiplex-PAP, the use of one universal-quencher but of multiple fluorophores simplifies the primer design and improves the amplification uniformity.

### 2) Pyrophosphorolysis Activated Fluorescence

Within structure of a fluorophore-quencher-dual-labeled blocked primer, through a mechanism of fluorescence-resonance energy transfer (FRET) from the fluorophore to the quencher, the fluorophore is kept from emitting fluorescence.

In PAP amplification, once the fluorophore-quencher-dual-labeled blocked primer (Format I) anneals to its complementary template, pyrophosphorolysis removes the 3′ non-extendable, blocked nucleotide which is covalently attached with the quencher such as TATRA, releasing and thus releasing the fluorophore, such as FAM, from the quencher to emit fluorescence, and then polymerization extends the 3′ unblocked primer (FIG. 1A).

In addition, different fluorophore-quencher-dual-labeled blocked primers can have different fluorophores such as FAM, HEX, and TET but one universal quencher, such as TAMRA, to differentiate multiple templates in a single reaction.

The number of released fluorophore is equal to the number of fluorophore-quencher-dual-labeled blocked primer exhausted, and also to the number of amplified product, a 1:1:1 molar relationship. Furthermore, each of them is directly proportional to intensity of the emitted fluorescence. These are true to both single and multiple fluorophore-quencher-dual-labeled blocked primers.

As for format II of fluorophore-quencher-dual-labeled blocked primer, the mechanism also includes pyrophosphorolysis to release the fluorophore to emit fluorescence (FIG. 1B).

Besides other advantages, pyrophosphorolysis activated fluorescence is specific to the amplified product because it needs pyrophosphorolysis, an enzyme catalyzed chemical reaction involving covalent bond change, to release and thus activate the fluorophore. It is also efficient because the number of the released fluorophore accumulates exponentially in PAP amplification.

Furthermore, the differences between pyrophosphorolysis activated fluorescence (Format I) and Taqman probe (Holland et al., 1991) are compared in Table 1.

TABLE 1
Comparison between pyrophosphorolysis activated fluorescence and Taqman probe

| | Pyrophosphorolysis activated fluorescence | Taqman probe |
|---|---|---|
| Associated technology | PAP amplification | PCR amplification |
| Assay composition for | Two blocked primers only | Two regular primers and one probe |
| one amplicon | | |
| Structure to generate | A fluorophore-quencher dual- | A fluorophore-quencher dual-labeled probe |
| fluorescence | labeled blocked primer | |
| Mechanism to | Pyrophosphorolysis of quencher at the 3′ | Hydrolysis of fluorophore at the 5′ |
| generate fluorescence | blocked nucleotide of the primer | nucleotide of the probe |
| Multiplexing | Yes | Yes |
| Polymerase activities | Pyrophosphorolysis and 5′-3′ | 5′-3′ polymerization and 5′-3′ |
| required | polymerization activities | exonuclease activities |
| Fluorescence | Exponential with cycles, the | Super-exponential with cycles, the |
| accumulation mode | number of released fluorophore = | number of released fluorophore ≠ the |
| | the number of amplified product | number of amplified product |

### Example 1 of Materials and Method

### Preparation of Primers

3′ ddCMP blocked primers were chemically synthesized in 3′-5′ direction and purified by HPLC by Integrated DNA Technologies.

3′ ddAMP, ddTMP and ddGMP blocked primers were synthesized enzymatically by adding ddATP, ddTTP and ddGTP to the 3′ ends of oligodeoxynucleotides by terminal transferase (Liu and Sommer, 2000; Liu and Sommer, 2002).

5′ end or internal single dye (FAM, HEX) labeled primers were chemically synthesized in 3′-5′ direction and purified by HPLC by Integrated DNA Technologies.

Rhodamine dye labeled dideoxynucleotide analogs or terminators of TAMRA-ddATP, TAMRA-ddUTP, TAMRA-ddGTP, and TAMRA-ddCTP were purchased from PerkinElmer Life Sciences, in which TAMRA is attached to the base of the nucleotide through a covalent bond. Then they were added to the 3′ ends of the single dye labeled primers by terminal transferase to synthesize dual dye labeled blocked primers.

Finally they were purified by 7M urea/16% polyacrylamide gel electrophoresis. The amount of each recovered primer was determined by UV absorbance at 260 nm.

### Preparation of Templates

Genomic DNA was extracted from blood white cells using QIAamp Blood Mini Kit according to Qiagen's protocol.

Recombinant plasmid DNA was constructed by inserting into pUC57 vector a 300 bp HIV-1 target DNA segment which was chemically synthesized. After transformed into E. coli, the recombinant plasmid DNA was extracted using QIAamp Plasmid Mini Kit according to Qiagen's protocol.

The eluted DNA was dissolved in TE buffer (10 mM Tris-HCl, 0.1 mM EDTA, pH8.0) and its amount was determined by UV absorbance at 260 nm.

### PAP Reaction

Unless stated otherwise, each reaction mixture of 20 μl contained 88 mM Tris-HCl (pH 8.0 at 25° C.), 10 mM (NH₄)₂SO₄, 2 mM MgCl₂, 25 μM each dNTP (dATP, dTTP, dGTP and dCTP), 0.1 μM each of primers, 150 μM Na₄PPᵢ, starting DNA template of the wildtype genomic DNA and/or plasmid DNA, and 1 units of polymerase for PAP amplification.

### Thermocycling and Fluorescence Detection

A Bio-Rad CFX96 real-time PCR detection system was used for amplification. A cycling entailed 96° C. for 12 seconds, 60° C. for 30 seconds, 64° C. for 30 seconds, and 68° C. for 30 seconds for a total of 45 cycles. A denaturing step of 96° C. for 2 min was added before the first cycle.

Analysis mode: fluorophore, Baseline setting: baseline subtracted curve fit, Threshold cycle (Ct) determination: single threshold, Baseline method: Auto calculated, Threshold setting: auto calculated. Filter mode was selected for FAM and/or HEX fluorescence detections. Ct value was thus measured for each reaction which is proportional to the amount of amplified product in the early exponential phase of amplification.

### Example 2 of Singleplex and Multiplex-PAP Assays of the GNAS and HIV Genes

Singleplex and multiplex PAP assays of the GNAS and HIV genes were used to demonstrate how pyrophosphorolysis activated fluorescence works.

GNAS and HIV genes are chosen as target and control because HIV-1 viral DNA can integrate into human genome and thus their detection from genomic DNA isolated from blood white cells is a medical need.

A singleplex PAP assay was tested with a forward fluorophore-quencher-dual-labeled blocked primer (SEQ ID 1) and a reverse blocked primer (SEQ ID 2) in the GNAS gene (Table 2). The fluorophore-quencher-dual-labeled blocked primer (SEQ ID 1) contains a HEX fluorophore covalently attached to a dAMP at the 5′ end, and a TAMRA quencher covalently attached to a ddCMP at the 3′ end. When the GNAS gene was amplified form 100,000 copies of the human wildtype genomic DNA (i.e., 330 ng), a HEX fluorescence signal was generated and measured at Ct 20.5 (FIG. 2A), the cycle number at which the HEX fluorescence signal crossed the threshold of 87 fluorescence units. In addition for the non-template control, no HEX fluorescence signal was seen above the threshold, showing the specificity.

Another singleplex PAP assay was tested with a forward fluorophore-quencher-dual-labeled blocked primer (SEQ ID 3) and a reverse blocked primer (SEQ ID 5) in the HIV gene (Table 2). The fluorophore-quencher-dual-labeled blocked primer contains a FAM fluorophore covalently attached to a dTMP at the 11th nucleotide from the 5′ end (SEQ ID 3) and a TAMRA quencher covalently attached to a ddUMP at the 3′ end. When the HIV gene was amplified form 100 copies of the HIV plasmid DNA template, a FAM fluorescence signal was generated and measured at Ct 32.2 (FIG. 2B), the cycle number at which the FAM fluorescence signal crossed the threshold of 122 fluorescence units. Another forward fluorophore-quencher-dual-labeled blocked primer (SEQ ID 4) was also tested with a similar result, which contains a FAM fluorophore covalently attached to a dTMP at the 5′ end and a TAMRA quencher covalently attached to a ddUMP at the 3′ end. In addition, no FAM fluorescence signal was seen above the threshold in the non-template control, showing the specificity.

A multiplex PAP assay was tested that contains the GNAS and HIV assays in a reaction. The GNAS assay contains the forward fluorophore-quencher-dual-labeled blocked primer (SEQ ID 1) and the reverse blocked primer (SEQ ID 2), and the HIV assay contains the forward fluorophore-quencher-dual-labeled blocked primer (SEQ ID 3) and the reverse blocked primer (SEQ ID 5) (Table 2). To simulate the copy ratio of the GNAS to HIV templates in medical condition, 100,000 copies of the human wildtype genomic DNA (i.e., 330 ng) and 100 copies of the HIV plasmid DNA template were added to a reaction. When the GNAS and HIV genes were amplified, HEX and FAM fluorescence signals were generated and measured at Ct 21.4 and 32.0, respectively (FIG. 2C). Another forward fluorophore-quencher-dual-labeled blocked primer (SEQ ID 4) was also tested with similar results. In addition, the non-template control did not generated HEX or FAM fluorescence signals above the thresholds, showing the specificities.

Thus, pyrophosphorolysis activated fluorescence was developed to measure PAP amplification in singleplex and multiplex PAP assays.

TABLE 2
Primers of the GNAS and HIV genes

| | | | 5′ end or | | | Fluorophore- |
|---|---|---|---|---|---|---|
| | | | internal | | | quencher |
| | Primer | Sequence (5′ to 3′) | fluoro- | 3′ end | 3′ end | dual- |
| Gene | pair | (SEQ ID NO)^{ab} | phore^{b} | quencher^{b} | blocker^{a} | labeled |
| GNAS | Forward | 5′HEX- | 5′ end HEX | TAMRA | ddCMP | Yes |
| gene | | ACTCTGAGCCCTCTTT | | | | |
| | | CCAAACTACT- | | | | |
| | | TAMRA-ddC3′ (SEQ ID | | | | |
| | | 1) | | | | |
| | Reverse | 5′GTCTCAAAGATTCCA | | | ddCMP | No |
| | | GAAGTCAGGACAddC3′ | | | | |
| | | (SEQ ID 2) | | | | |
| HIV | Forward | 5′ TTGGAGGACA(FAM- | Internal | TAMRA | ddUMP | Yes |
| gene | | T)CAAGCAGCCATGCA | FAM | | | |
| | | AA-TAMRA-ddU3′ (SEQ | | | | |
| | | ID 3) | | | | |
| | Forward | 5′FAM- | 5′ end FAM | TAMRA | ddUMP | Yes |
| | | TTGGAGGACATCAAG | | | | |
| | | CAGCCATGCAAA- | | | | |
| | | TAMRA-ddU3′ (SEQ ID | | | | |
| | | 4) | | | | |
| | Reverse | 5′ TGCTATGTCAGTTCC | | | ddTMP | No |
| | | CCTTGGTTCTCddT3′ | | | | |
| | | (SEQ ID 5) | | | | |

Footnotes of Table 2.
^{a}Each primer contains a 3′ non-extendable, blocked nucleotide, such as ddCMP at the 3′ end, and matches its starting template along the length.
^{b}Fluorophore-quencher dual-labeled blocked primers (SEQ ID 1, 3 and 4) contain a fluorophore covalently attached to a nucleotide in the internal nucleotide or at the 5′ end, and a quencher covalently attached to a dideoxynucleotide at the 3′ end. The types of the fluorophore and quencher are also indicated.

### Example 3 of Singleplex and Multiplex Delayed-PAP Assays of the GNAS and HIV Genes

Delayed-PAP was developed by introducing an artificial mutation into the 3′ region of a blocked primer, which can delay the product accumulation to a later time or cycle in PAP amplification (U.S. patent application 62/683,725). Pyrophosphorolysis activated fluorescence was further demonstrated in such delayed-PAP assays.

A singleplex GNAS delayed-PAP assay was tested with a forward fluorophore-quencher-dual-labeled blocked primer (SEQ ID 1) (Table 2) and a reverse-M1 primer which contains a G to T 3′-artificial mutation at the 5^{th} nucleotide from the 3′ end (SEQ ID 6) (Table 3). Another singleplex GNAS delayed-PAP assay was also tested with the forward fluorophore-quencher-dual-labeled blocked primer (SEQ ID 1) and another reverse-M2 primer which contains a C to A 3′-artificial mutation at the 3rd nucleotide from the 3′ end (SEQ ID 7) (Table 3). When the GNAS gene was amplified from 100,000 copies of the human wildtype genomic DNA (i.e., 330 ng), HEX fluorescence signals were generated and measured at Ct 21.8 and 27.6 for the two GNAS assays. Compared with the GNAS regular-PAP assay (SEQ ID 1 and 2) in Example 2, Ct values of the two GNAS assays (SEQ ID 1 and 6, and SEQ ID 1 and 7) were delayed by up to 7.1 cycles.

A multiplex PAP assay was tested that contains the GNAS delayed-PAP assay (SEQ ID 1 and 6) and the HIV PAP assay (SEQ ID 3 and 5) in a reaction. Another multiplex PAP assay was also tested that contains the GNAS delayed-PAP assays (SEQ ID 1 and 6) and the HIV PAP assay (SEQ ID 1 and 7). 100,000 copies of the human wildtype genomic DNA (i.e., 330 ng) and 100 copies of the HIV template were amplified in a reaction. For the GNAS gene, HEX fluorescence signals were generated and measured at Ct 22.0 and 27.9 for the two GNAS delayed-PAP assays, respectively. Compared with the GNAS regular-assay (SEQ ID 1 and 2) in Example 2, Ct values of the two GNAS delayed-PAP assays (SEQ ID 1 and 6, and SEQ ID 1 and 7) were delayed by up to 7.4 cycles. On the other hand, for the HIV gene, FAM fluorescence signals were generated and measured at Ct 33.4 and 32.7 for the two reactions of the HIV assay (SEQ ID 3 and 5), respectively, substantially consistent with Example 2.

Thus, pyrophosphorolysis activated fluorescence to measure PAP amplification of nucleic acid was demonstrated in singleplex and multiplex delayed-PAP assays.

TABLE 3
Primers of the GNAS gene containing 3′-artificial mutations

| | | 3′-artificial mutation^{c} | |
|---|---|---|---|
| Primer^{a} | Sequence (5′ to 3′) (SEQ ID NO) | Type | From the 3′ end |
| Reverse-M1 | 5′ GTCTCAAAGATTCCAGAAGTCAG | G to T | 5nt |
| | TACAddC3′ (SEQ ID 6)^{b} | | |
| Reverse-M2 | 5′ GTCTCAAAGATTCCAGAAGTCAG | C to A | 3nt |
| | GAAAddC3′ (SEQ ID 7) | | |

Footnotes of Table 3.
^{a}For the GNAS gene, the two blocked primers for delayed PAP had 3′-artificial-mutations introduced to mismatch to their starting templates. Each of reverse-M1 and M2 primers (SEQ ID 6 and 7) was paired with a fluorophore-quencher dual-labeled blocked primer (SEQ ID 1).
^{b}For this reverse-M1 primer, an artificial mutation T is indicated as bold and underlined case. In addition, ddC means dideoxynucleotide C at the 3′end.
^{c}For the 3′-artificial-mutation, the type and location from the 3′ end are also indicated in the 3′ region. The nucleotide at the 3′ end is assigned as 1.

### REFERENCE

Deutscher M P, Kornberg A. 1969. Enzymatic synthesis of deoxyribonucleic acid. 28. The pyrophosphate exchange and pyrophosphorolysis reactions of deoxyribonucleic acid polymerase. J Biol Chem 244(11):3019-28.
Holland P M, Abramson R D, Watson R, Gelfand D H. 1991. Detection of specific polymerase chain reaction product by utilizing the 5′-3′ exonuclease activity of Thermus aquaticus DNA polymerase. Proc. Natl. Acad. Sci. USA 88(16):7276-7280.
Liu Q, Nguyen V Q, Li X, Sommer S S. 2006. Multiplex dosage pyrophosphorolysis-activated polymerization: application to the detection of heterozygous deletions. Biotechniques 40(5):661-8.
Liu Q, Sommer S S. 2000. Pyrophosphorolysis-activated polymerization (PAP): application to allele-specific amplification. Biotechniques 29(5): 1072-1080.
Liu Q, Sommer S S. 2002. Pyrophosphorolysis-activatable oligonucleotides may facilitate detection of rare alleles, mutation scanning and analysis of chromatin structures. Nucleic Acids Res 30(2):598-604.
Liu Q, Sommer S S. 2004a. Detection of extremely rare alleles by bidirectional pyrophosphorolysis-activated polymerization allele-specific amplification (Bi-PAP-A): measurement of mutation load in mammalian tissues. Biotechniques 36(1): 156-66.
Liu Q, Sommer S S. 2004b. PAP: detection of ultra rare mutations depends on P* oligonucleotides: “sleeping beauties” awakened by the kiss of pyrophosphorolysis. Hum Mutat 23(5):426-36.
Liu Q, Sommer S S. 2004c. Pyrophosphorolysis by Type II DNA polymerases: implications for pyrophosphorolysis-activated polymerization. Anal Biochem 324(1):22-8.
Modrich P. 1987. DNA mismatch correction. Annu Rev Biochem 56:435-66.

1. A pair of forward and reverse blocked primers for pyrophosphorolysis activated polymerization, comprising a 3′ blocked primer to be complementary to a template, wherein a quencher is covalently attached to a nucleotide in the internal region or at the 5′ end, and a fluorophore is covalently attached to the base of a non-extendable, blocked nucleotide that lacks the 3′ hydroxyl group and is located at the 3′ end of the primer, and wherein once the primer anneals to its template, the 3′ non-extendable, blocked nucleotide attached with the fluorophore is removed from the 3′ end of the primer by pyrophosphorolysis catalyzed by a polymerase to generate two products of 1) a 3′ unblocked primer which is covalently labeled with the quencher in the internal region or at the 5′ end, and 2) a corresponding blocked nucleoside triphosphate which base is covalently labeled with the fluorophore, thus the fluorophore emits a fluorescence signal in PAP amplification.

2. The pair of forward and reverse blocked primers for pyrophosphorolysis activated polymerization of claim 1, wherein the 3′ blocked primer has a FAM (carboxyfluorescein) or HEX (hexachlorofluorescein) fluorophore attached to the blocked nucleotide at the 3′ end, and a TAMRA (carboxytetramethylrhodamine) quencher to the nucleotide in the internal region or at the 5′ end.

3. A plurality of 3′ blocked primers for multiplex pyrophosphorolysis activated polymerization comprising:
a) a first 3′ blocked primer to be complementary to a first template, wherein a universal quencher is covalently attached to a nucleotide in the internal region or at the 5′ end, and a first fluorophore is covalently attached to the base of a first non-extendable, blocked nucleotide which lacks the 3′ hydroxyl group and is located at the 3′ end of the first primer, and wherein once the first primer anneals to the first template, the first 3′ non-extendable, blocked nucleotide attached with the first fluorophore is removed from the 3′ end of the first primer by pyrophosphorolysis catalyzed by a polymerase to generate two products of 1) a 3′ unblocked primer which is covalently labeled with the universal quencher in the internal region or at the 5′ end, and 2) a corresponding blocked nucleoside triphosphate which base is covalently labeled with the first fluorophore, thus the first fluorophore emits a fluorescence signal in PAP amplification, and
b) a second 3′ blocked primer to be complementary to a second template, wherein the universal quencher is covalently attached to a nucleotide in the internal region or at the 5′ end, a second fluorophore is covalently attached to the base of a second non-extendable, blocked nucleotide which lacks the 3′ hydroxyl group and is located at the 3′ end of the second primer, and wherein once the second primer anneals to the second template, the second 3′ non-extendable, blocked nucleotide covalently attached with the second fluorophore is removed from the 3′ end of the second primer by pyrophosphorolysis catalyzed by the polymerase to generate two products of 1) a 3′ unblocked primer which is covalently labeled with the universal quencher in the internal region or at the 5′ end, and 2) a corresponding blocked nucleoside triphosphate which base is covalently labeled with the second fluorophore, thus the second fluorophore emits another fluorescence signal in PAP amplification.

4. The plurality of 3′ blocked primers for multiplex pyrophosphorolysis activated polymerization of claim 3, wherein the first blocked primer has a universal TAMRA (carboxytetramethylrhodamine) quencher attached to the nucleotide in the internal region or at the 5′ end and a FAM (carboxyfluorescein) fluorophore attached to the first blocked nucleotide at the 3′ end, and wherein the second blocked primer has the same universal TAMRA (carboxytetramethylrhodamine) quencher attached to the nucleotide in the internal region or at the 5′ end and a HEX (hexachlorofluorescein) fluorophore attached to the second blocked nucleotide at the 3′ end.

5. The plurality of 3′ blocked primers for multiplex pyrophosphorolysis activated polymerization of claim 3, wherein the first primer has the same universal quencher as the second primer, but the first primer has a different fluorophore compared with the second primer.

6. The plurality of 3′ blocked primers for multiplex pyrophosphorolysis activated polymerization of claim 3, wherein the first template is an internal control and the second template is a target.

7. The plurality of 3′ blocked primers for multiplex pyrophosphorolysis activated polymerization of claim 3, wherein emission spectrums of the fluorophores overlap an absorbance spectrum of the universal quencher.

8. The plurality of 3′ blocked primers for multiplex pyrophosphorolysis activated polymerization of claim 3, wherein the quencher is located 30 bases or less from the 3′ ends.

9. The plurality of 3′ blocked primers for multiplex pyrophosphorolysis activated polymerization of claim 3, wherein the blocked nucleotides are dideoxynucleotides or dideoxynucleotides.

10. A method for multiplex pyrophosphorolysis activated polymerization with plurality of blocked primers, comprising:
a) a first 3′ blocked primer to be complementary to a first template, in which a universal quencher is covalently attached to a nucleotide in the internal region or at the 5′ end, and a first fluorophore is covalently attached to the base of a first non-extendable, blocked nucleotide that lacks the 3′ hydroxyl group and is located at the 3′ end of the first primer,
b) wherein once the first primer anneals to the first template, the first 3′ non-extendable, blocked nucleotide attached with the first fluorophore is removed from the 3′ end of the first primer by pyrophosphorolysis catalyzed by a polymerase to generate two products of 1) a 3′ unblocked primer which is covalently labeled with the universal quencher in the internal region or at the 5′ end, and 2) a corresponding blocked nucleoside triphosphate which base is covalently labeled with the first fluorophore, thus the first fluorophore emits a fluorescence signal in PAP amplification,
c) a second 3′ blocked primer to be complementary to a second template, in which the universal quencher is covalently attached to a nucleotide in the internal region or at the 5′ end, a second fluorophore is covalently attached to the base of a second non-extendable, blocked nucleotide that lacks the 3′ hydroxyl group and is located at the 3′ end of the second primer, and
d) wherein once the second primer anneals to the second template, the second 3′ non-extendable, blocked nucleotide covalently attached with the second fluorophore is removed from the 3′ end of the second primer by pyrophosphorolysis catalyzed by the polymerase to generate two products of 1) a 3′ unblocked primer which is covalently labeled with the universal quencher in the internal region or at the 5′ end, and 2) a corresponding blocked nucleoside triphosphate which base is covalently labeled with the second fluorophore, thus the second fluorophore emits another fluorescence signal in PAP amplification.
